(19) United States
(12) Patent Application Publication
(10) Pub. No.: US 2008/0021103 A1
(43) Pub. Date: Jan. 24, 2008

(54) Mucoadhesive composition

(75) Inventors: Khawla Abdullah ABU-IZZA, West Chester, PA (US); Krishna Pattabi RAMAN, Bear, DE (US); Vincent Hon-Kin Li, Mechanicsville, VA (US); Sauwaluxana TONGAREE, Nutley, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/903,458

(22) Filed: Sep. 21, 2007

Related U.S. Application Data
(63) Continuation of application No. 10/173,251, filed on Jun. 17, 2002, now abandoned.
(60) Provisional application No. 60/298,508, filed on Jun. 15, 2001.

(51) Int. Cl. A61K 47/30; A61K 31/165; A61K 31/192; A61K 31/24

(52) U.S. Cl. 514/536; 514/570; 514/617; 514/772.3

(57) ABSTRACT

The invention is directed to a topical medicament-containing composition which exhibits excellent bioadhesive properties suitable for application to the human body, including to mucous membranes.

### FIELD OF INVENTION

This invention relates to an improved topical composition. In particular, the composition exhibits bioadhesive properties which make it suitable for application to the human body, including to mucous membranes. Such properties of the composition keep the composition in contact with the affected area for several hours, resulting in a period of prolonged administration of any medicament(s) contained therein.

### BACKGROUND OF THE INVENTION

A large percentage of the population is at one time or another in need of a topically applied medicament. However, adhesion to the surface of the human body has proven to be difficult to achieve due to the presence of body oils and perspiration. Mucous membranes have been found to be a particular difficult area to achieve long-term adhesion due to the presence of increased levels of moisture and the sensitivity of such areas to chemical irritants.

U.S. Pat. No. 5,081,158 discloses a long lasting film forming composition for mucosal application. The film forming polymer used is hydroxypropyl cellulose. Organic acids such as tannic acid and salicylic acid are contained in the composition to esterify the polymer. Boric acid is further present to aid in polymer crosslinking. When the composition is applied to oral mucosa, a tough film forms upon evaporation of the composition's solvent. However, the composition suffers from the presence of residual levels of free organic acids that do not participate in the esterification or crosslinking reactions. These acids often cause irritation, especially if in contact with mucous membranes. Another difficulty is the limited bioadhesion exhibited by the polymer. While the polymer film may maintain its physical strength for several hours, it often detaches from the mucosa long before the film fails.

U.S. Pat. No. 5,885,611 discloses an oral gel that forms an adherent oral bandage upon evaporation of the composition's solvent. Ethylcellulose is utilized as the film-forming polymer.

While ethylcellulose forms a very film having good strength, the film does not exhibit good bioadhesion qualities. Therefore, such films typically do not adhere very long, often failing in less than one hour

### OBJECTS OF THE INVENTION

It is an object of the present invention to develop a topical medicament-containing composition exhibiting good film-forming properties and good bioadhesive properties, even to mucous membranes.

It is still another object of the present invention to develop a topical composition which does not contain potential irritants such as unreacted organic acids.

It is still a further object of the present invention to develop a topical medicament-containing composition in which increased levels of the active medicament component may be incorporated.

### SUMMARY OF THE INVENTION

The present invention is directed to a topical medicament-containing composition. The composition comprises at least one medicament, a film forming composition comprising polymethacrylate, a mucoadhesive polymer and a solvent therefore. It has been found that through use of the claimed composition increased levels of the medicament may be administered.

### DESCRIPTION OF THE INVENTION

This invention relates to an improved medicament-containing composition comprising at least one medicament and a film forming mucoadhesive vehicle comprising polymethacrylate, a solvent therefore and a mucoadhesive polymer.

The identity of the medicament component(s) is not critical. It must be suitable for topical application and stable within the claimed composition. It may be selected from a host of recognized medicaments heretofore used in topical applications. For instance, the medicament may be selected from one or more of the following general categories: local anesthetics, topical analgesics, antiseptics/antibacterial agents, anti-inflammatory agents, antiviral agents, antifungal agents and mixtures thereof. Examples of local anesthetics include benzocaine, benzyl alcohol and lidocaine. Examples of topical analgesics include methyl salicylate, menthol, thymol and camphor Examples of antiseptic/antibacterial compounds include benzalkonium chloride, benzethonium chloride, and cetylpyridinium Examples of anti-inflammatory components include ibuprofen and ketoprofen. Examples of antiviral components include acyclovir. Examples of antifungal agents include miconazole and clotrimazole.

The concentration of the medicament(s) will, of course, vary according to their approved dosing levels and their solubility in the vehicle component of the claimed invention. For instance, benzocaine, a preferred topical anesthetic, may be present in amounts generally ranging from about 5% to about 25% on a weight percentage basis relative to the total composition. More preferably, benzocaine may be present in amounts ranging from about 10% to about 20% on the same basis. Most preferably, benzocaine may be present in an amount on about 20% on the same basis.

Lidocaine, another preferred topical anesthetic, may be present in amounts generally ranging from about 2% to about 10% on a weight percentage basis relative to the total composition. More preferably, lidocaine may be present in amounts ranging from about 2% to about 5% on the same basis. Most preferably, lidocaine may be present in an amount on about 5% on the same basis.

Benzalkonium chloride, a topical antiseptic may be present in amounts generally ranging from about 0.005% to about 0.15% on a weight percentage basis relative to the total composition. More preferably, it may be present in amounts ranging from about 0.01% to about 0.02% on the same basis.

Ibuprofen, an antiinflammatory may be present in amounts generally ranging from about 1% to about 20% on a weight percentage basis relative to the total composition. More preferably, it may be present in amounts ranging from about 5% to about 10% on the same basis.

As stated above, the vehicle of the claimed composition comprises polymethacrylate as the film-forming polymer. The polmethacrylate polymer may be present in amounts generally ranging from about 0.1 to 35% by weight relative to the total composition. Preferably, it is present in amounts ranging from about 10 to about 25% by weight on the-same basis. Most preferably, it is present in an amount of about 15% on the same basis. Preferred in the practice of the present invention is the use of Eudragit® RS PO, a product manufactured by Rohm, which is a copolymer of acrylate and methacrylates with quaternary ammonium group as a functional group. Eudragit® RS PO is insoluble in water. As a result, when the composition is applied to the oral mucosa and allowed to dry, the formed film will not dissolve in saliva and therefore lasts longer.

The vehicle also contains a solvent suitable for the polymethacrylate polymer. The choice of the solvent is not critical so long as it is suitable both for use with polymethacrylate and in topical pharmaceutical compositions. If the medication is intended for use on canker sores, the solvent should be suitable for use on oral mucosa. The use of ethyl alcohol is preferred. The solvent may be present in amounts generally ranging from about 20 to 95% by weight relative to the total composition. Preferably, it is present in amounts ranging from about 30-70% by weight on the same basis. Most preferably, it is present in an amount of about 55-65% on the same basis.

The claimed composition also contains at least one mucoadhesive polymer that also acts as a viscosity modifying agent. Examples of such viscosity modifying agents include acrylic acid polymers (such as Carbopl® 940, also known as Carbomer® 940, Carbopol 934P and Carbopol® 980, products of BF Goodrich), methyl vinyl/maleic acid copolymers (such as Gantrez® S-97, a product of Internationl Specialty Products), polyvinyl pyrrolidone also known as povidone (such as Plasdone® K-90, a product of Internationl Specialty Products). These polymers impart relatively high viscosity to the composition at relatively low concentrations. They may therefore be incorporated into the claimed composition is amounts ranging from about 0.01% to about 10% by weight relative to the total composition. Preferably, they are present in gels amounts ranging from about 1-5% by weight on the same basis. Most preferably, they are present in an amount of about 3% on the same basis. These viscosity modifying agents further act to improve the film adhesion of the composition to mucous membranes. The preferred mucoadhesive/viscosity modifying agent is Carbopol. The preferred grades are Carbopol® 934P, 971 P, 974P and 980 and the most preferred grade of Carbopol® is Carbopol® 980. The preferred level of Carbopol® 980 is 2-3% by weight of the total composition. Most preferably, it is used in an amount of about 2.5% on the same basis.

The claimed composition may be formulated as either a liquid or as a gel. If a liquid formulation is desired a relatively low concentration (such as 0.1-1%) of the mucoadhesive/viscosity modifying agent can be used. If a gel formulation is desired, a higher concentration (e.g. 1.5-4%) of the suitable viscosity modifying/mucoadhesive agent can be incorporated into the polymethacrylate/solvent vehicle for gel formation.

The claimed composition may further comprise excipients such as plasticizers, flavorings, sweeteners and/or colorants. Examples of plasticizers include triethyl citrate, polyethylene glycol and glycerin. Such plasticizers are present in the composition in amounts generally ranging from about 1% to about 10% by weight relative to the total composition. For example, glycerine can be present in the amount of 1-5% by weight of the composition, preferably in the amount of 2.5% on the same basis. Polyethylene glycol and triethyl citrate can be used in the amount of about 5 to about 12%, preferably in the amount of 8%.

The claimed composition is prepared by conventional techniques wherein the polymethacrylate component, the mucoadhesive polymer (e.g., Carbopol), optional ingredients (such as sweeteners, and/or colorants) and the medicament(s) are dissolved in the solvent and optional liquid ingredients such as plasticizers and flavors. The resulting mixture is subjected to a conventional mixing operation to evenly disperse the components.

While the viscosity of the claimed composition may vary widely depending upon its final intended application, it is preferred that it possess a viscosity of about 5000 to about 25000 cps. This can be attained through variation in the amount of the mucoadhesive polymer, which also serves as a viscosity builder.

Upon application, the solvent rapidly evaporates leaving a tough adhesive film that both administers the medicament contained therein and acts as a protective barrier against irritants such as food and beverages. The composition may be applied on an “as needed” basis. As a general guideline, it should be administered about every 4-6 hours. The claimed composition further acts as a sustained release dosage form for the medicament, thereby prolonging the treatment intervals.

It has further been found during the development of the claimed invention that the solubility of the medicament in the vehicle is enhanced. As such, higher concentrations of the medicament can be incorporated into the vehicle relative to that which is soluble in the solvent alone. While not wishing to be bound to any theory, it appears that the polymer may be acting as a stabilizer for the medicament in solution.

The following Examples are offered to illustrate the claimed method and its practice. They should not however be construed in any way as a limitation to the scope of the present

### EXAMPLE 1

The mixture having the following composition was prepared:

| Ingredient | % wt./wt. of composition |
|---|---|
| Benzocaine | 20.0 |
| Carbomer 980 (acylic acid | 3.5 |
| Polymer) | |
| Eudragit RS PO (poly- | 20.0 |
| Methacrylate) | |
| Triethyl citrate | 8.0 |
| Glycerin | 3.0 |
| Saccharin ([sweetener] | 0.25 |
| FD&C Red #40 (colorant) | 0.01 |
| Flavor | 2.0 |
| Ethyl alcohol | 43.2 |

The resulting mixture was placed in a stainless steel beaker and subjected to mixing conditions through the use of an air driven mixer for a duration of approximately 2 hours. A smooth, clear gel having a viscosity of about 17,000 cps resulted.

### EXAMPLE 2

The mixture having the following composition was prepared:

| Ingredient | % wt./wt. of composition |
|---|---|
| Benzocaine | 15.0 |
| Carbomer 980 (acylic acid | 2.5 |
| Polymer) | |
| Eudragit RS PO (poly- | 15.0 |
| Methacrylate) | |
| Glycerin | 3.0 |
| Saccharine ([sweetener] | 0.25 |
| Opatint (colorant) | 0.01 |
| Flavor | 2.0 |
| Ethyl alcohol | 62.0 |

The resulting mixture was placed in a stainless steel beaker and subjected to mixing conditions through the use of an air driven mixer for a duration of 2 hours. A smooth, clear gel having a viscosity of about 20,000 cps resulted.

### EXAMPLE 3

The mixture having the following composition was prepared:

| Ingredient | % wt./wt. of composition |
|---|---|
| Benzocaine | 20.0 |
| Carbomer 974 (acylic acid | 3.0 |
| Polymer) | |
| Eudragit RS PO (poly- | 15.0 |
| Methacrylate) | |
| Gantrez S-97 (copolymer of | 1.5 |
| methyl vinyl/maleic | |
| acid) | |
| Glycerin | 3.0 |
| Saccharine ([sweetener]) | 0.25 |
| FD&C Red #40 (colorant) | 0.01 |
| Flavor | 2.0 |
| Ethyl alcohol | 48.3 |

The resulting mixture was placed in a stainless steel beaker and subjected to mixing conditions through the use of an air driven mixer for a duration of 2 hours. A slightly hazy, smooth gel having a viscosity of about 10,000 cps resulted.

### EXAMPLE 4

The mixture having the following composition was prepared:

| Ingredient | % wt./wt. of composition |
|---|---|
| Benzocaine | 15.0 |
| Carbomer 934 (acylic acid | 2.0 |
| polymer) | |
| Eudragit RS PO (poly- | 15.0 |
| methacrylate) | |
| Gantrez S-97 (copolymer of | 2.0 |
| methyl vinyl/maleic | |
| acid) | |
| ethylcellulose N-22 | 2.0 |
| glycerine | 5.0 |
| Saccharine ([?]) | 0.25 |
| FD&C Red #40 (colorant) | 0.01 |
| Flavor | 2.0 |
| Ethyl alcohol | 56.8 |

The resulting mixture was placed in a stainless steel beaker and subjected to mixing conditions through the use of an air driven mixer for a duration of 2 hours. A slightly hazy, smooth gel having a viscosity of about 11,000 cps resulted.

### EXAMPLE 5

| | |
|---|---|
| Benzocaine | 20.0% |
| Carbomer 980 (acylic acid | 2.5 |
| Polymer) | |
| Eudragit RS PO (poly- | 15.0 |
| Methacrylate) | |
| Glycerin | 3.0 |
| Saccharine (sweetener) | 0.25 |
| Opatint (colorant) | 0.01 |
| Flavor | 2.0 |
| Ethyl alcohol | 57.2 |

The preparation was conducted as in the previous examples. A slightly hazy gel with viscosity of approximately

### EXAMPLE 6

A sensory study was conducted using 19 healthy volunteers to compare the composition of Example #1 with a commercial product marketed under the Zilactin-B® trademark. Each sample was applied to the oral mucosa of the volunteers in the same way and allowed to dry for 30 seconds. The mucosa was then visually inspected every hour for film presence. Six hours after application, the film of Example 1 was still present in 70% of the subjects while the Zilactin® film was present in only 57% of the subjects.

### EXAMPLE 7

An animal study was conducted using 6 New Zealand white rabbits to compare the efficacy of the composition of Example 2 with two (2) commercial formulations, Zilactin-B® and Orajel® Ultra. 0.05 ml of each product was applied to the oral mucosa of 6 New Zealand white rabbits for five successive days. The animals were inspected every 30 minutes for film presence. The average duration for example 2 was 2.34 hours. The average duration for Zilactin®-B and Oragel® Ultra was 1.59 hours and 1.67 hours, respectively.

1. A topical medicament-containing composition for application to the oral mucosa comprising:
a. at least one medicament component in an effective concentration;
b. a film forming composition comprising polymethacrylate and a solvent therefore; and mucoadhesive polymer,
wherein the polymethacrylate is insoluble in water and is present in an amount ranging from about 5 to about 25 percent by weight of the total composition, and further the composition is either a gel or a liquid.

2-31. (canceled)

32. The composition of claim 1 wherein the polymethacrylate is present in the film-forming composition in an amount of about 15 percent by weight of the total composition.

33. The composition of claim 1 wherein the polymethacrylate solvent is present in an amount ranging from about 20 to about 95 percent by weight of the total composition.

34. The composition of claim 1 wherein the polymethacrylate solvent is present in an amount ranging from about 30 to about 70 percent by weight of the total composition.

35. The composition of claim 1 wherein the polymethacrylate solvent is present in an amount ranging from about 55 to about 65 percent by weight of the total composition.

36. The composition of claim 1 wherein the polymethacrylate solvent is ethanol.

37. The composition of claim 1 wherein the polymethacrylate solvent is ethanol and is present in an amount ranging from about 20 to about 95 percent by weight of the total composition.

38. The composition of claim 36 wherein the polymethacrylate solvent is ethanol and is present in an amount ranging from about 30 to about 70 percent by weight of the total composition.

39. The composition of claim 36 wherein the polymethacrylate solvent is ethanol and is present in an amount ranging from about 55 to about 65 percent by weight of the total composition.

40. The composition of claim 1 wherein the medicament is selected from the group consisting of local anesthetics, topical anesthetics, antiseptic/antibacterial agents, anti-inflammatory agents, antiviral agents, antifungal agents and mixtures thereof.

41. The composition of claim 40 wherein the medicament is selected from the group consisting of benzocaine, lidocaine and ibuprofen.

42. The composition of claim 41 wherein benzocaine is present in amounts ranging from about 5 to about 25 percent by weight relative to the weight of the total composition.

43. The composition of claim 42 wherein benzocaine is present in an amount of about 20 percent by weight relative to the weight of the total composition.

44. The composition of claim 1 wherein the mucoadhesive polymer is selected from acrylic acid polymers, methyl vinyl/maleic acid polymers and polyvinyl polymers.

45. The composition of claim 44 wherein the mucoadhesive polymers are present in the composition in amounts ranging from about 0.01 to about 10 percent by weight.

46. The composition of claim 44 wherein the mucoadhesive polymers are present in the composition in amounts ranging from about 1 to about 5 percent by weight.

47. The composition of claim 44 wherein the mucoadhesive polymers are present in the composition in an amount of about 2.5 percent by weight.

48. The composition of claim 1 wherein the viscosity of the composition ranges from about 5000 to about 25000 cps.

49. A method of administering a medicament to an individual comprising topically applying to said individual the composition of claim 1.

50. The method of claim 49 wherein the composition is topically applied to the mucosa of the individual.
